(19) United States
(12) Patent Application Publication
(10) Pub. No.: US 2020/0305374 A1
(43) Pub. Date: Oct. 1, 2020

(54) METHOD FOR INDUCING ACTIVE RESPONSE OF PLANTS

(71) Applicant: BEIJING XINNA INTERNATIONAL NEW MATERIALS TECHNOLOGY CO., LTD., Beijing (CN)

(72) Inventors: Gong JIN, Beijing (CN); Zhijiang GUO, Beijing (CN); Jiangping TU, Beijing (CN); Jianping XIE, Beijing (CN); Xiang FANG, Beijing (CN); Lijie CI, Beijing (CN); Ligang ZUO, Beijing (CN); Zhian LI, Beijing (CN)

(73) Assignee: BEIJING XINNA INTERNATIONAL NEW MATERIALS TECHNOLOGY CO., LTD., Beijing (CN)

(21) Appl. No.: 16/759,604

(22) PCT Filed: Nov. 1, 2017
(86) PCT No.: PCT/CN2017/108932

§ 371 Date: Apr. 27, 2020

(51) Int. Cl. A01H 3/00

(52) U.S. Cl. CPC A01H 3/00

(57) ABSTRACT

The present invention provides a method, which is used with trace amount, can initiate an active endogenous response of plants, and can obtain expected effects such as growth acceleration and yield increase of the plants. The present invention further provides preparation of a signal carrier material for implementing the method. In the method, by utilizing genetic information stored in the plants, the plants can adapt to an environment through a signal which makes the plants mistakenly perceive, thereby achieving an expected objective. The method is suitable for planting of crops in regions with poor natural conditions or severe climate changes.

### TECHNICAL FIELD

The present invention belongs to the field of biotechnology, and particularly relates to a method for inducing plants to actively adapt to an environment and a signal carrier for implementing the method. The method replaces the traditional method at present in which the environment is modified to adapt to crop production by enabling the plants to actively adapt to environmental changes.

### BACKGROUND

The chemical evolution hypothesis that the primitive life evolved from inorganic small molecules to organic small molecules, biomacromolecules and multimolecular systems in the primitive environment on earth has more scientific basis than other hypotheses. The present invention is not concerned with the origin and evolution of species, but more with the impact of environmental changes and signals on the evolution of life during the evolution of life from the primitive environment (3.5 to 4.0 billion years ago) to the current earth's climate environment, so as to find new methods and ideas of crop production to realize the sustainable development of environment and agriculture at the same time.

In 9,000-10,000 B.C., human beings had obtained plants output by using and domesticating plants. Taking rice as an example, compared with human domesticated and planted rice, wild rice is capable of resisting and adapting to environmental changes. But domestication is a long process of following natural evolution. In order to meet the current demand of human beings for plants output, at present, an idea is to modify the soil environment to adapt to crop growth by, for example, irrigation and fertilization, thereby adapting the environment to meet needs of modern crop cultivation. However, excessive human intervention of the environment has had an irreversible impact on the current natural environment. Another idea is to accelerating the adaptation of crops to the environment, primarily by using transgenic technology to recombine crop genes to generate new traits desired by humans and to cultivate new varieties. As artificially produced varieties, transgenic crops violate the law of natural evolution and are regarded as alien species that do not exist in nature. Generally speaking, the threat or danger caused by alien species to the environment or biological diversity will continue for a long period of time, such as 10 years or more. Since the commercial planting of the transgenic crops has only been for 5-6 years, some potential risks may not show in such a short period of time. Thus, the transgenic technology is relatively technology that remains to be seen, and a long time process will be required to evaluate its long-term safety.

The idea of the present invention is to enable a crop to adapt to the environment, and essentially differs from the transgenic technology as follows. By utilizing genetic information stored in an early evolution process of plants, the signal carrier simulates an environmental change to make plants perceive such changes in environmental signals; and native genes of the plants produce different expression levels in different growth periods by endogenous signal transduction of the plants as well as autonomous regulation of molecules, cells, and biology. In this way, the growth, development, accumulation and reproduction process of the plants are accelerated, so that beneficial effects expected by humans such as resistance improvement, growth acceleration and yield increase are achieved.

As survival and reproduction are the most basic attributes of all living things, when the external living condition changes, plants can autonomously perform targeted gene expression of genetic information in the evolution process by sensing the environmental signals, and generates and reflects change in the plants endogenous signal and a biological response.

It is worth noticing that since the current climate environment is quite different from the primitive environmental conditions of the origin of life, under simple treatment of temperature, light and water vapor, plants cannot sense such conditions to activate the complex genetic code in the evolution process. Or, the plants actively respond to such conditions, but the response does not conform to the expectations of human beings. For example, it is reported that in contact with an exogenous signal material through an artificially direct or indirect way, plants can recognize the exogenous signal and respond actively, premature senescence of the plants is accelerated as the biochemical metabolism in vivo is enhanced while starting the endogenous signal. For instance, inoculation with some domesticated micro viruses may induce the plants to activate a metabolic mechanism based on jasmonic acid pathway to accelerate the growth of the plants in the early stage, but plants die prematurely due to excessive metabolism.

In summary, although the theory of plants adaptation to the environment is feasible, it is necessary to find a suitable signal carrier to implement the method. The chemical evolution hypothesis, particularly, the evolution process of the primitive life from inorganic small molecules to organic small molecules, the biomacromolecules and the multimolecular systems may provide inspirations such as follows:

Firstly, under a mild condition, the transformation of an inorganic substance into an organic substance may be used as a primitive environmental signal. Miller's experiment proves that in a primitive extreme environment, a simple combination of simple inorganics of carbon, hydrogen, oxygen and nitrogen can be converted into an organic substance, proving qualitative change from inanimate substances to living organisms through electronic information transfer between atoms or molecules. It should be noted that the continuous discharge in the Miller's experiment are not suitable to the existing form of current life forms. For example, the existing species will die under continuous electric shock. Subsequent modified experiments prove that it is more suitable for the transformation from the inorganic substance to organic small molecules in accordance with the temperature, light and other conditions suitable for the plants growth. The patents CN106431508A by the applicant proves that an organic small molecule can be produced at a voltage of 3 to 5V when graphite and water are provided with carbon, hydrogen and oxygen.

Secondly, the transformation from organic small molecules into biological macromolecules serves as the basis of the environmental signal. Protein and nucleic acid are the material basis of life, and especially, the interaction of organic small molecules in the environment can produce condensation or polymerization reaction to start the further transport and metabolism of life. Patent Application No. PCT/CN 2017/086086 of the applicant proves that the basic units of proteins, namely amino acids (such as glycine and alanine) will emerge under the presence of nitrogen in the environment.

The present invention mainly describes that the signal carrier can change gene expression levels of plants in different growth phases.

### SUMMARY

An object of the present invention is to provide a method for initiating an active response of plants. In the method, by utilizing genetic information stored in an early evolution process of the plants, an environmental signal that makes the plants mistake for a growth acceleration need is artificially created, so that the plants actively senses an environmental change to activate the adaptability, thereby achieving beneficial effects expected by human beings such as growth acceleration, biomass and yield increase, and resistance and quality improvement. The method replaces the current strategy of adapting an environment to the plants made by human beings, so as to reduce ecological risks caused by excessive demand of human beings on plants output.

Another object of the present invention is to solve a negative problem which is caused by contact between the plants and an exogenous signal material through an artificially direct or indirect way in the prior art.

The theoretical basis of the present invention is as follows: in an evolution process of primitive life from inorganic small molecules to organic small molecules, biomacromolecules and multimolecular systems, the evolution information of life responding to changes in environmental conditions such as cold, heat, and freezing in a form of gene coding is stored in the plants. Although the current earth environment has lost the basic conditions of the origin of life, survival and reproduction are still the most basic attributes of all living things, and genetic information in the evolution process is still stored in dominant or recessive genetic codes of the plants. Thus, when an external living condition changes, the plants can autonomously perform targeted gene expression by sensing an environmental signal, and generates a change in plants endogenous signal and a biological response. Such change in the endogenous signal can be characterized and analyzed by testing expression levels of plants genes in different stages.

The present invention is implemented by the following technical solutions.

A method for inducing an active response of plants, the method using signal carrier material as an exogenous signal carrier for the plants; and

the plants sensing, contacting or capturing the exogenous signal carrier, and the plants perceiving an exogenous signal carried by the exogenous signal carrier and making an active response.

The plants actively responding to the exogenous signal perceived, manifested by detection of a targeted parameter through observation, genetic, physiological, cellular or phenotypic testing.

The active response to the exogenous signal perceived by the plants is embodied in a detected change in an expression level of an endogenous hormone in signal transduction in plants.

The change in the expression level of the endogenous hormone in the plants is embodied in a detected change in the expression level of the hormone autonomously and endogenously synthesized by the plants through genetic or physiological detection, wherein the hormone is at least one of five main endogenous hormones.

The active response to the exogenous signal perceived by the plants is embodied in a detected change in a genetic cell function of the plants, wherein the change in the genetic cell function of the plants is mainly embodied in changes of cells and organelles.

The changes of the cells and organelles comprise changes in at least one of chloroplast, mitochondrion, golgi complex and endoplasmic reticulum.

The active response to the exogenous signal perceived by the plants is embodied in a detected change in a genetic cell function of the plants, wherein the change in the genetic cell function of the plants is mainly embodied in a change in a catalytic activity or an electron transfer activity.

The change in the genetic cell function of the plants further comprises a change related to an immune response.

The active response to the exogenous signal perceived by the plants is embodied in a detected change in a genetic biological pathway of the plants, wherein the change is mainly embodied in a change in growth, transport, reproduction or biological rhythm of the plants.

The change further comprises a change in auxiliary anabolism associated with the growth, transport, reproduction or biological rhythm of the plants.

The active response to the exogenous signal perceived by the plants is embodied in a phenotypic characteristic of at least one of biological regulation processes of resistance increase, growth acceleration and yield increase of the plants.

The signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition.

The mild formation condition means that none of the temperature, light, electric field and magnetic field endangers the life of a current organism.

The present disclosure has the following advantageous effects.

The method and the signal source carrier for implementing the method provided by the present invention are characterized by simulating the mild condition in the early stage of life and transforming the simple inorganic substance into the organic small molecules. In the method, by utilizing the genetic information stored in the early evolution process of the plants, the environmental change is simulated on the basis of the signal carrier; the plants is caused to sense the change of the environmental signal; and through the endogenous signal transduction of the plants as well as the autonomous regulation of molecules, cells, and biology, the growth, development, accumulation and reproduction process of the plants is accelerated while expressing resistance genes, so that beneficial effects expected by humans such as resistance improvement, growth acceleration and yield increase are achieved. The method foregoes the traditional approach of adapting the environment to the plants; by utilizing the genetic information stored in the plants themselves, the plants can adapt to the environment through the signal enabling the plants to mistakenly sense, so that objectives expected by the human beings are realized. The method has the characteristics of green and environmental protection, is suitable for planting of crops in regions with poor natural conditions or severe climate changes, and has effects of a relatively stable production and a high yield.

In addition, as opposed to conserved genes recording the origin of species, the plasticity of genes, evolved from biological genes to adapt to the environment, and refers to gene expressions responding to changes of the external environment. Based on this, different from the current separate regulation of adapting an environment condition to plants, the present invention avoids uncontrollable risks caused by the separate regulation, and invents, starting from an environmental signal, the environmental signal that makes the plants mistakenly sense a growth acceleration need to induce organisms to start gene memory in the form of the signal, so that the plants can adapt to environmental changes, and effects expected by humans such as resistance improvement, biomass increase, growth acceleration and yield increase are realized. Thus, the plants can adapt to the environment, and the consumption of pesticides, antibiotics, fertilizers and the like in agricultural planting is reduced, ensuring safety, green and environmental protection.

### BRIEF DESCRIPTION OF THE DRAWINGS

FIG. 1 is a schematic diagram showing plants capturing a signal carrier material;

FIG. 2A is a schematic diagram of gene analysis and GO;

FIG. 2B is a schematic diagram of GO analysis;

FIG. 3 is a comparison diagram of changes of both cells and organelles;

FIG. 4A is a comparison plot of hormone change in plants root system;

FIG. 4B is a comparison plot of hormone change in plants leaf;

FIG. 5 is a comparison of Arabidopsis thaliana vs. Arabidopsis thaliana with a biological response; and

FIG. 6 is a schematic diagram of an active response of plants according to the present invention.

### DETAILED DESCRIPTION

The following embodiments are employed to illustrate the technical solutions of the present invention. These embodiments are merely illustrative, are only intended to describe and explain the technical solutions of the present invention, and should not be construed as limiting the technical solutions of the present invention.

In the present application, signal carrier material is prepared first. The signal carrier material of the present application is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition.

In the present application, the mild formation conditions mean that none of temperature, light, electric field and magnetic field endangers the life of a current organism. For example, the maximum temperature cannot exceed a highest tolerance temperature of current organisms, and the lowest temperature cannot exceed a lowest tolerance temperature of current organisms. Similarly, none of the light, the electric field and the magnetic field exceeds a tolerance of the current organism.

Therefore, none of organic substances prepared from inorganic substances under parameters or conditions beyond tolerable conditions of the current organisms is within the protection scope of the present application, and is comparable with the protection scope of the present application.

For example, inorganic graphite and water are transformed into an organic CHO skeleton to form a bioactive substance such as amino acid.

In the present application, plants are in contact with a signal carrier material by means of sensing, capturing, contacting, adhering fixation, or the like. In the present application, the contact between plants and the signal carrier material is observable and detectable.

The present application provides a method for inducing an active response of plants. As shown in FIG. 6, a signal carrier material serves as an exogenous signal carrier of the plants. In the present application, the signal carrier material is released into the growth environment of the plants in a trace amount. Excluding the loss amount, the plants can sense the amount of the signal carrier material applied. Applying the signal carrier material into the environment of the plants is not limited to application to the soil, and may be application to leaves, water or a nutrient solution of the plants, and may further comprise any other application manners through which the signal carrier material can be put in contact with the plants.

The plants sense, contact or capture the exogenous signal carrier, and perceive an exogenous signal carried by the exogenous signal carrier and make an active response.

The active response to the exogenous signal perceived by the plants is manifested in a detected targeted parameter through visual observation, or genetic, physiological, cellular or phenotypic detection method.

The active response to the exogenous signal perceived by the plants is embodied in a detected change in an expression level of an endogenous hormone in signal transduction in plants. The signal transduction means that a change in the endogenous hormone, such as salicylic acid, auxin, cytokinin, abscisic acid or ethylene, can be detected.

The change in the expression level of the endogenous hormone in the plants is embodied in a detected change in the expression level of the hormone autonomously and endogenously synthesized by the plants through a genetic or physiological detection, wherein the hormone is at least one of five main endogenous hormones.

The active response to the exogenous signal perceived by the plants is embodied in a detected change in a genetic cell function of the plants, wherein the change in the genetic cell function of the plants is mainly embodied in changes of cells and organelles.

The changes of the cells and organelles comprise changes in at least one of chloroplast, mitochondrion, golgi complex and endoplasmic reticulum, and refer to the detected changes of the cells and the organelles, which include the size and the number of layers, as well as structure changes of internal organelles (comprising the chloroplast, photosynthetic antenna protein, mitochondria, golgi complex, etc.).

The active response to the exogenous signal perceived by the plants is embodied in a detected change in a genetic cell function of the plants, wherein the change in the genetic cell function of the plants is mainly embodied in a change in a catalytic activity or an electron transfer activity.

The change in the genetic cell function of the plants further comprises a change related to an immune response. A change process of the immune response of the plants means that encoding information stored in the plants may be implemented through, for example, a catalyst or an electronic carrier, and by detects overexpression, namely, an active response, of a gene encoded by such gene information.

The active response to the exogenous signal perceived by the plants is embodied in a detected change in a genetic biological pathway of the plants, wherein the change is mainly embodied in a change in growth, transport, reproduction or biological rhythm of the plants.

The change further comprises a change in auxiliary anabolism associated with the growth, transport, reproduction or biological rhythm of the plants, and refers to that changes in the activity of ATP and related enzymes that can be detected.

The active response to the exogenous signal perceived by the plants is embodied in a phenotypic characteristic of at least one of biological regulation processes of resistance increase, growth acceleration and yield increase of the plants. The biological regulation process comprises a detected change in a physiological metabolic pathway, such as dry mater synthesis, a nutrient absorption ability and a root vitality, and further comprises the phenotypic characteristic differences such as a biomass change, a growth rate change and a yield change.

The principle of the active response of the plants in the technical solution is as follows.

Genes have the conservatism of recording the origin of species and the plasticity of adapting to environmental changes. In particular, after hundreds of millions of years of changes, the species have evolved a set of gene information that can adapt to external stresses (such as cold, heat, drought, flooding, diseases and pests), etc. Thus, by mobilizing and utilizing information stored by genes to adapt to environmental changes, a condition that can be perceived by the plants is provided in the presence of a signal provided artificially, so as to trigger defense and immune coding stored in the genes. Therefore, the core principle of the present invention is to provide a method and design concept that can trigger the active defense response of the plants, thereby accelerating the the growth and the evolution of the plants, and maintaining the fundamental attribute of reproduction of the biological genes.

### Embodiments

The signal carrier material is prepared by the following manner. Graphite, water and nitrogen in the air serve as raw materials to provide sources of carbon, hydrogen, oxygen and nitrogen required for the synthesis of the signal carrier material. At room temperature, only 3V DC is applied to obtain the signal carrier material with carbon, hydrogen, oxygen and nitrogen as main components. The signal carrier material has obvious biological properties. For example, in the signal carrier material, at least one kind of amino acids can be detected through mass spectrometry or other methods. The carrier further has other biochemical properties such as oxidation reduction, complexation, and chelation, which can be described by different parameters such as pH values and isoelectric points.

In another embodiment of the present application, graphite and water which serve as raw materials and an added sulfur element provide sources of carbon, hydrogen, oxygen and sulfur required for the synthesis of the signal carrier material. At a normal temperature, only a DC voltage less than 5V is applied to obtain the signal carrier material with carbon, hydrogen, oxygen and sulfur as main components. The signal carrier material has obvious biological properties.

Similarly, in another embodiment of the present application, graphite, water and air which serve as raw materials and an added sulfur element provide sources of carbon, hydrogen, oxygen, nitrogen and sulfur required for the synthesis of the signal carrier material. At the normal temperature, only a DC voltage less than 5V is applied to obtain the signal carrier material with carbon, hydrogen, oxygen, nitrogen and sulfur as main components. The signal carrier material has obvious biological properties.

In another embodiment of the present application, graphite and water serve as raw materials to provide sources of carbon, hydrogen and oxygen required for the synthesis of the signal carrier material. At the normal temperature, only a DC voltage less than 5V is applied to obtain the signal carrier material with carbon, hydrogen and oxygen as main components. The signal carrier material has obvious biological properties.

In another embodiment of the present application, graphite, water and air which serve as raw materials and an added phosphorus element and sulfur element provide sources of carbon, hydrogen, oxygen, nitrogen, phosphorus and sulfur required for the synthesis of the signal carrier material. At the normal temperature, only a DC voltage less than 5V is applied to obtain the signal carrier material with carbon, hydrogen, oxygen, nitrogen, phosphorus and sulfur as main components. The signal carrier material has obvious biological properties.

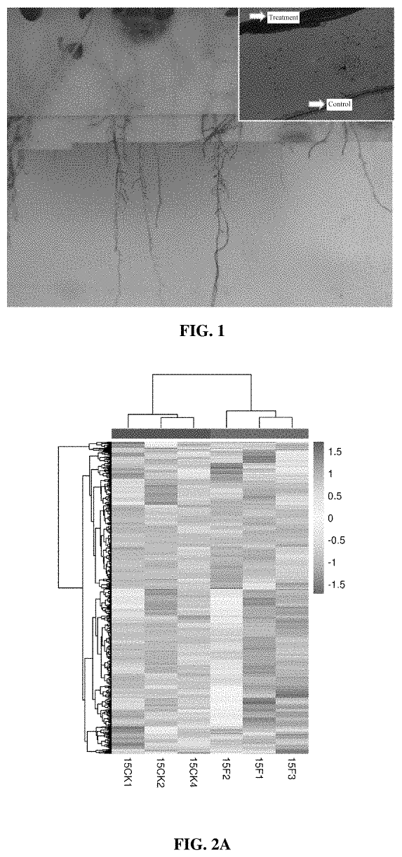

1.32 mg of the above signal carrier material is added into a cultivation environment of a biological model plants, Arabidopsis thaliana. FIG. 1 shows a process that a root system captures and fixate the signal carrier onto the surface of the root system, and further shows a difference between the root system with the captured and fixated signal carrier and an observed control optical structure slice. It is obvious that the root system with the captured signal carrier is blacker, and the root system cannot be observed clearly due to the attachment.

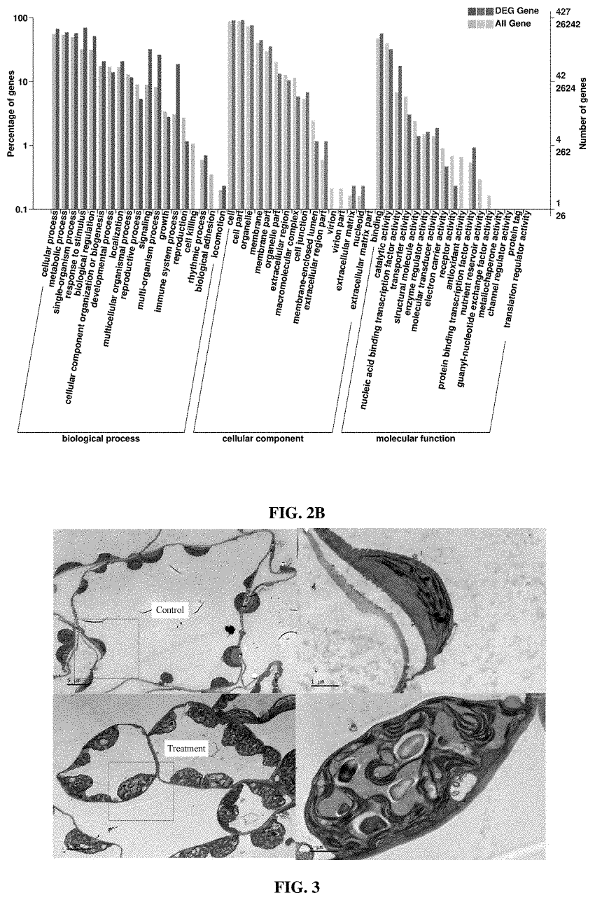

FIG. 2A illustrates overall differences of transcriptomic differential genes and clusters between three groups of controls and three groups of treated Arabidopsis thaliana after capturing the signal carrier, and colors are used to distinguish the results of gene expressions. The results indicate that Arabidopsis thaliana responds to the stimulation of the exogenous signal carried by the signal carrier substance. The exhibited transcriptome difference further indicates that the method for initiating the active response of the plants through the exogenous gene stimulation is feasible. FIG. 2B further illustrates functions initiated by the signal carrier material, i.e., the influence on the existence of the gene, from the molecular function, cell component and biological process. Through the comparison of the different genes, it is indicated that after the signal carrier material is located in the cell membrane, the plants treats the signal carrier material as an exogenous stimulus firstly; and the immune defense of the plants is initiated through molecular function effects such as catalysis and electron transfer, and is manifested by increase of metabolic activity. More importantly, plants signal transduction pathway is activated. Through the cascade amplification of the signal, it is manifested by a change in synthesis of organelles at the cellular level, and is manifested by a change in a physiological process such as growth, transport or breeding at the biological level.

FIG. 3 includes further illustration of the changes in cells and cell components. With respect to leaves at the same positions, in the Arabidopsis thaliana treated with the signal carrier, the numbers of chloroplasts and chloroplast grana are changed, and the granum lamellas are curled, which indicate that the cells and the organelles respond to the stimulation of this signal carrier. This figure further explains the cellular composition and biological process enhanced by the photosynthesis in FIG. 2B.

This signal carrier material activates a signal pathway of calcium-dependent protein kinase (CDPK) and regulates the response mechanisms comprising ROS pathway and the like. Meanwhile, the signal carrier material further provides evidence of initiation of metabolic pathways such as jasmonic acid, proving that the plants can perceive the stimulation of this signal carrier material and can produce signal cascade amplification to initiate an immune metabolic pathway.

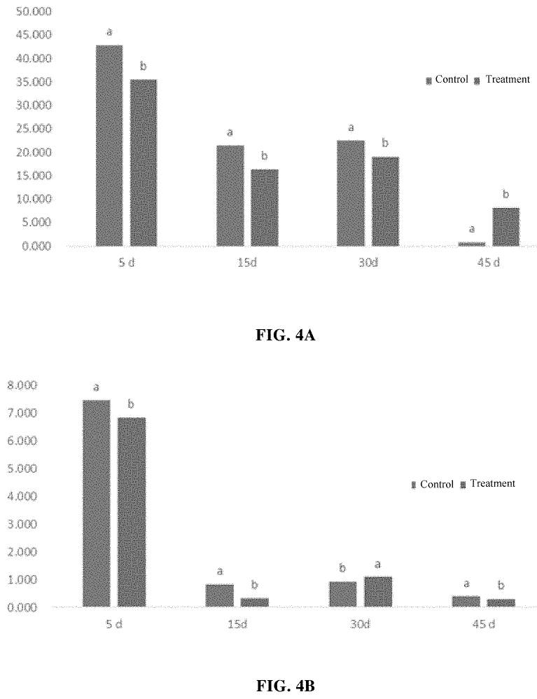

FIGS. 4A and 4B show changes in the root system and the jasmonic acid content level of the leaf in the model plants Arabidopsis thaliana treated with the signal carrier material, so as to illustrate the effect of the signal carrier material on the endogenous signal transduction in the plants.

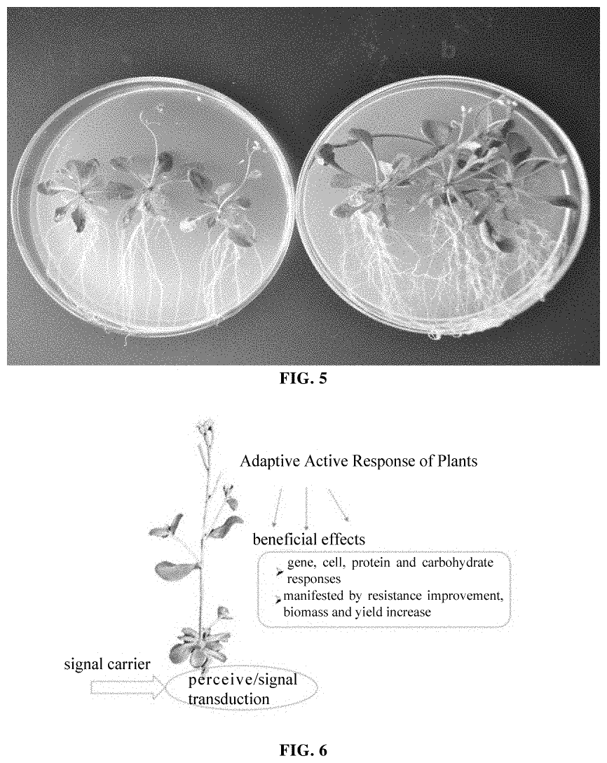

FIG. 5 shows a phenotypic response, in which the left of the FIG. 5 shows the growth of the control, and the right of the FIG. 5 shows that after the release of 1.32 mg/L of the signal carrier, Arabidopsis thaliana shows an increased biomass, a faster growth rate and the like to further illustrate the processes of FIGS. 1 to 4.

The rice field experiment in a saline alkali land in Zhenlai County, Baicheng City, Jilin Province of China in 2017 with this method and the involved carrier shows the following results. A control group and an experimental group are set for rice cultivation in soda meadow saline-alkali soil with a soil pH of 9.0-10, a salt content of 1%, a soil organic matter content of 2%, an alkaline-hydrolyzable nitrogen content of 80 ppm, a rapid available phosphorus content of 14 ppm and a rapid available potassium content of 95 ppm; in the experimental group, the application amount of the signal carrier of the present invention is 0.3% of that of a local fertilizer; yields and soils are tested simultaneously; the results show that the signal carrier does not change the physical and chemical properties of the saline-alkali land, the yield of the control group without using the method is 327.8 kglmu, and the yield of the rice in the experimental group using the method is 509.6 kg/mu, which is 55.44% higher than that of the control group. Thus, the problems of a low yield and no panicle of the rice caused by soil salination and alkalization for a long time in this region are effectively solved.

In the foregoing, only some illustrative embodiments of the present invention are described for illustration. It is apparent to those skilled in the art that modifications of the above-described embodiments may be made in various ways without departing from the spirit and scope of the present invention. Therefore, the foregoing drawings and description are illustrative in nature and should not be interpreted to limit the scope of the present invention as defined by the claims.

1. A method for inducing an active response of plants, characterized in that the method uses a signal carrier material as an exogenous signal carrier for the plants by utilizing genetic information stored in an early evolution process of the plants, and artificially creating an environmental condition that makes the plants mistake for a growth acceleration requirement;
the plants perceive an exogenous signal carried by the exogenous signal carrier by sensing or capturing the exogenous signal carrier, so that the plants perceive an environment change to accelerate growth and reproduction, and make an active response to the perception.

2. The method for inducing the active response of the plants according to claim 1, characterized in that the active response to the exogenous signal perceived by the plants is manifested by a detected targeted parameter through an observation, or a genetic, physiological, cellular or phenotypic detection method.

3. The method for inducing the active response of the plants according to claim 1, characterized in that the active response to the exogenous signal perceived by the plants is embodied in a detected change in an expression level of an endogenous hormone in signal transduction in plants.

4. The method for inducing the active response of the plants according to claim 3, characterized in that the change in the expression level of the endogenous hormone in the plants is embodied in a detected change in the expression level of the hormone autonomously and endogenously synthesized by the plants through a genetic or physiological detection, wherein the hormone is at least one of five main endogenous hormones.

5. The method for inducing the active response of the plants according to claim 1, characterized in that the active response to the exogenous signal perceived by the plants is embodied in a detected change in a genetic cell function of the plants, and the change in the genetic cell function of the plants is mainly embodied in changes of cells and organelles.

6. The method for inducing the active response of the plants according to claim 5, characterized in that the changes of the cells and organelles comprise changes in at least one of chloroplast, mitochondrion, golgi complex and endoplasmic reticulum.

7. The method for inducing the active response of the plants according to claim 1, characterized in that the active response to the exogenous signal perceived by the plants is embodied in a detected change in a genetic cell function of the plants, and the change in the genetic cell function of the plants is mainly embodied in a change in a catalytic activity or an electron transfer activity.

8. The method for inducing the active response of the plants according to claim 7, characterized in that the change in the genetic cell function of the plants further comprises a change related to an immune response.

9. The method for inducing the active response of the plants according to claim 1, characterized in that the active response to the exogenous signal perceived by the plants is embodied in a detected change in a genetic biological pathway of the plants, and the change is mainly embodied in a change in growth, transport, reproduction or biological rhythm of the plants.

10. The method for inducing the active response of the plants according to claim 9, characterized in that the change further comprises a change in auxiliary anabolism associated with the growth, transport, reproduction or biological rhythm of the plants.

11. The method for inducing the active response of the plants according to claim 1, characterized in that the active response to the exogenous signal perceived by the plants is embodied in a phenotypic characteristic of at least one of biological regulation processes of resistance increase, growth acceleration and yield increase of the plants.

12. The method for inducing the active response of the plants according to claim 1, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

13. The method for inducing the active response of the plants according to claim 12, characterized in that the mild formation condition means that none of temperature, light, electric field and magnetic field endangers the life of a current organism.

14. The method for inducing the active response of the plants according to claim 2, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

15. The method for inducing the active response of the plants according to claim 3, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

16. The method for inducing the active response of the plants according to claim 4, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

17. The method for inducing the active response of the plants according to claim 5, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

18. The method for inducing the active response of the plants according to claim 6, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

19. The method for inducing the active response of the plants according to claim 7, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.

20. The method for inducing the active response of the plants according to claim 8, wherein the signal carrier material is an organic substance that has a biological activity and that is transformed from an inorganic substance, and the organic substance is synthesized by carbon and hydrogen or oxygen, and at least one of nitrogen, sulfur and phosphorus in the inorganic substance under a mild formation condition; and effects expected by human beings such as growth acceleration, biomass and yield increase, and resistance and quality improvement are achieved after the plants perceive the signal carrier material.
